(19) United States
(12) Patent Application Publication
(10) Pub. No.: US 2018/0153620 A1
(43) Pub. Date: Jun. 7, 2018

(54) Spinal Navigation Method, Spinal Navigation System and Computer Program Product

(71) Applicant: Erasmus University Medical Center Rotterdam, Rotterdam (NL)

(72) Inventor: Sieger Leenstra, Rotterdam (NL)

(21) Appl. No.: 15/571,711

(22) PCT Filed: May 6, 2016
(86) PCT No.: PCT/NL2016/050327

§ 371 Date: Nov. 3, 2017

(30) Foreign Application Priority Data
May 6, 2015 (NL) 2014772

(51) Int. Cl. A61B 34/20; A61B 5/055; A61B 6/03; A61B 8/08; A61B 5/00; A61B 8/14; A61B 8/15; A61B 8/00; G06T 7/00; G06K 9/62; G06T 7/70

(52) U.S. Cl. CPC A61B 34/20; A61B 2090/364; A61B 6/032; A61B 8/0841; A61B 5/4566; A61B 8/14; A61B 8/15; A61B 8/4427; A61B 8/463; A61B 8/5261; G06T 7/0012; G06K 9/6202; G06T 7/70; A61B 2034/2063; A61B 2034/2065; A61B 5/055

(57) ABSTRACT

The invention relates to a spinal navigation method. The method comprises the steps of providing a MRI, X-ray or CT based two-dimensional image of the spine of a subject and generating an ultrasound two-dimensional image using an ultrasound imaging device on the spine of said subject. Further, the method comprises the steps of matching the ultrasound two-dimensional image to the MRI, X-ray or CT based two-dimensional image, and relating a pre-specified segment of a spinal profile in the MRI, X-ray or CT based two-dimensional image to a corresponding segment in the ultrasound two-dimensional image.

The invention relates to a spinal navigation method.

In clinical practice, it appears that in lumbar surgery, in 1-3% of the cases the point of surgical entry deviates from the intended location, leading to medical complications. The entry for lumbar surgery is generally localized by palpation of anatomical landmarks or by X-ray localization. However, palpation of anatomical landmarks can not always be performed in a correct way, e.g. due to fat tissue and/or deviating lumbar spines. On the other hand, X-ray devices are expensive. In addition, mobile X-ray devices are not easy to handle in operating rooms and expose patient and personnel to harmful X-ray beams.

Also other image generating devices generating relatively high resolution images such as MRI or CT devices are expensive and voluminous.

It is an object of the invention to provide a spinal navigation method that is accurate and can easily be applied in an operating room. Thereto, according to an aspect of the invention, a spinal navigation method is provided, comprising the steps of providing a MRI, X-ray or CT based two-dimensional image of the spine of a subject, generating an ultrasound two-dimensional image using an ultrasound imaging device on the spine of said subject, matching the ultrasound two-dimensional image to the MRI, X-ray or CT based two-dimensional image, and relating a pre-specified segment of a spinal profile on the MRI, X-ray or CT based two-dimensional image to a corresponding segment in the ultrasound two-dimensional image.

By applying an ultrasound imaging technique and matching the ultrasound two-dimensional image to a pre-generated or pre-operative MRI, X-ray or CT based two-dimensional image, a pre-specified segment of a spinal profile in the MRI, X-ray or CT based image can be related to a corresponding segment in the ultrasound two-dimensional image, thereby exploiting the relative accuracy of MRI, X-ray or CT technology with compact and easy to use ultrasound imaging devices, so that a voluminous and/or potentially harmful MRI, X-ray or CT mobile or stationary MRI, X-ray or CT image device is not needed anymore in the operating room, while on the other hand a desired accuracy in a pre-specified entry for spinal surgery such as lumbar surgery can be determined. When relating a pre-specified segment of a spinal profile in the MRI, X-ray or CT based image to a corresponding segment in the ultrasound two-dimensional image, a spinal contour in the ultrasound image can be recognized. As an example, a specific level, name or label of a spinal process in the ultrasound image can be recognized if a relation with the MRI, X-ray or CT based image is established.

The invention also relates to a spinal navigation system.

Further, the invention relates to a computer program product. A computer program product may comprise a set of computer executable instructions stored on a data carrier, such as a flash memory, a CD or a DVD. The set of computer executable instructions, which allow a programmable computer to carry out the method as defined above, may also be available for downloading from a remote server, for example via the Internet, e.g. as an app.

Other advantageous embodiments according to the invention are described in the following claims

By way of example only, embodiments of the present invention will now be described with reference to the accompanying figures in which

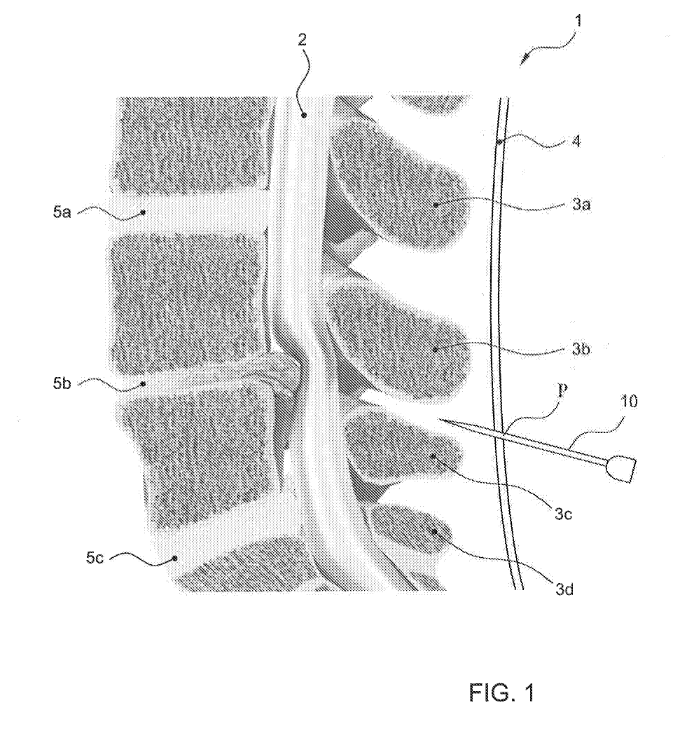

FIG. 1 shows a cross sectional schematic side view of the human lumbar spine;

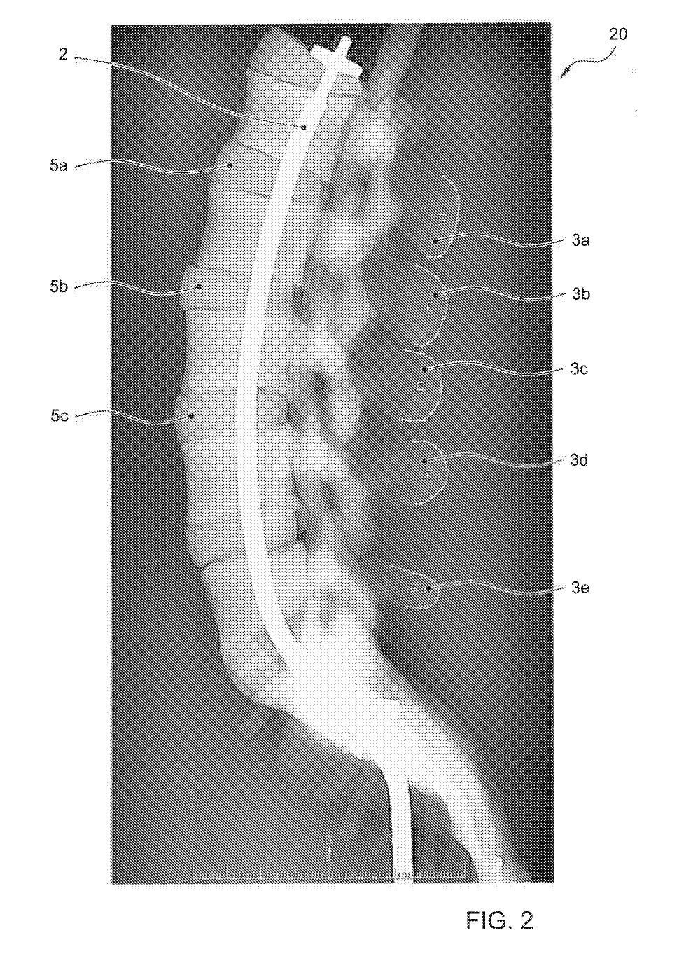

FIG. 2 shows an X-ray image of a lumbar spine;

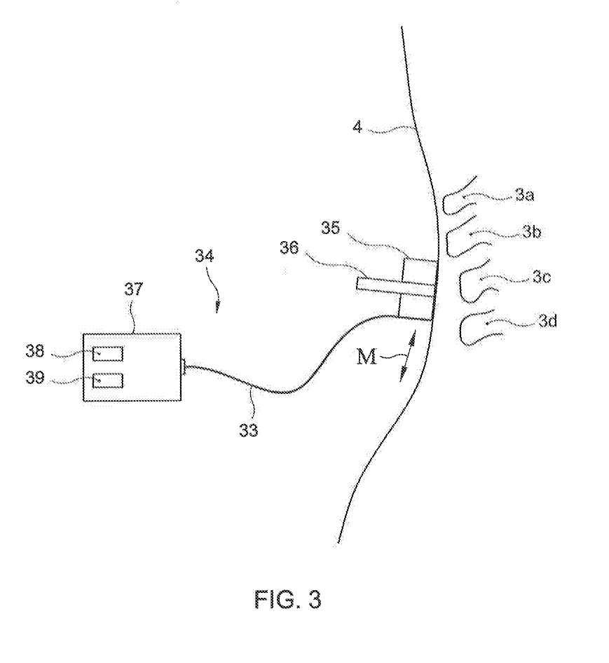

FIG. 3 shows a spinal navigation system according to the invention;

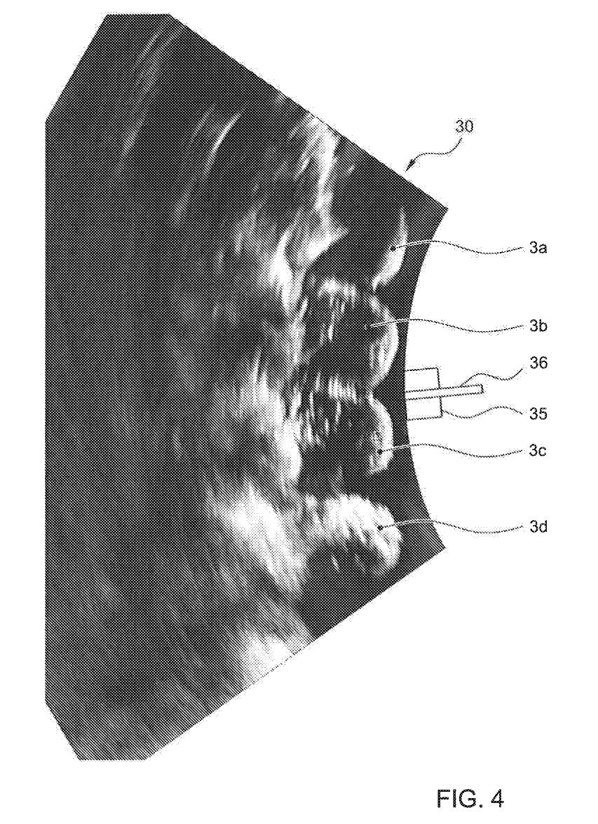

FIG. 4 shows an ultrasound image of the subject's spine;

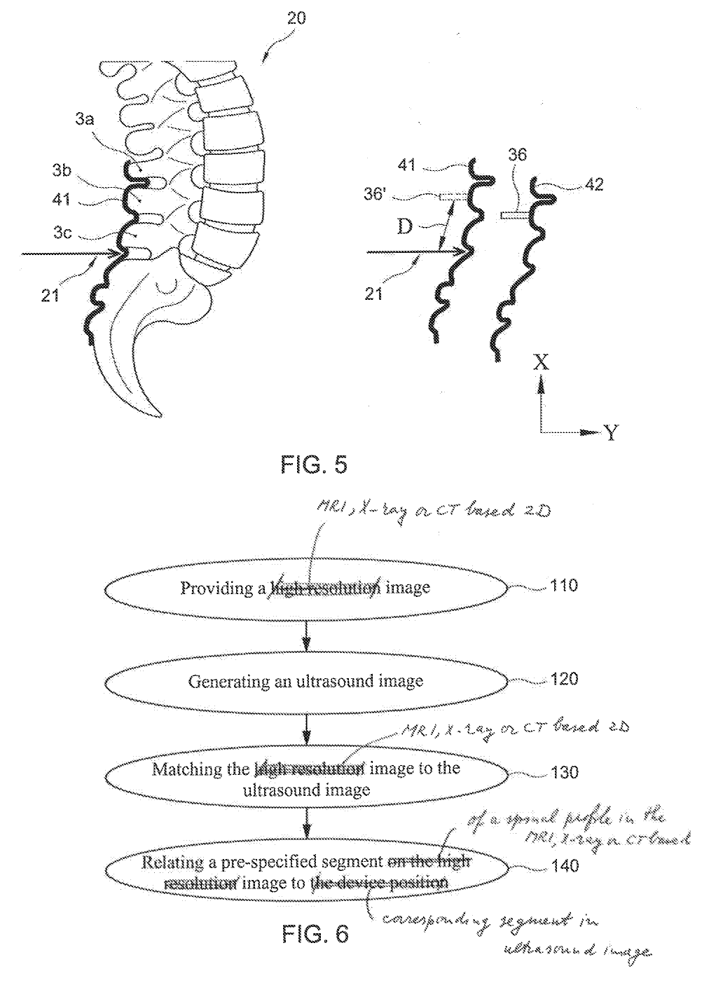

FIG. 5 shows a view wherein an X-ray image and an ultrasound image are matched;

FIG. 6 shows a flow chart of an embodiment of a method according to the invention.

The figures merely illustrate a preferred embodiment according to the invention. In the figures, the same reference numbers refer to equal or corresponding parts.

FIG. 1 shows a cross sectional schematic side view of the human lumbar spine 1 including a central nerve 2, more precisely lumbar sac with cauda equine, and a multiple number of spinal processus 3a-d extending from the central nerve 2 backwardly to the skin 4 of a subject's back. Further, the lumbar spine 1 includes a multiple number of intervertebral discs 5a-c extending away from the central nerve 2 opposite to the spinal processus 3a-d. Due to a slipped, herniated intervertebral disc 5b, the central nerve 2 is deformed and under static pressure causing physiological symptoms.

The slipped, herniated intervertebral disc can be treated by lumbar spine surgery, e.g. performed by neurosurgeons or orthopedic surgeons. The surgery is performed by approaching the lumbar spine 1 between two spinal processus 3, using a surgical device 10, in the shown side view between the L3 spinal processus 3b and the L4 spinal processus 3c. However, the lumbar spine 1 can also be entered between other spinal processus 3, e.g. between L4 and L5 or between L5 and S1, depending on the location of the slipped, herniated intervertebral disc 5b. For diagnostic purposes, an MRI image can be generated to analyse the position of the intervertebral discs and identify any Hernia Nuclei Pulposi HNP.

FIG. 2 shows an X-ray image 20 of a lumbar spine 1. The X-ray image is generated for spinal navigation to determine a point of entry P in the surgery process. It is noted that, as an alternative to the X-ray image, another two-dimensional image can be generated, e.g. based on CT imaging or MRI imaging such as 2D, 3D or 4D MRI. In FIG. 2, an X-ray image of a phantom lumbar spine 1 is shown provided with a bar through the center of the vertrebral bodies to keep it in place.

FIG. 3 shows a spinal navigation system 34 according to the invention. The system 34 comprises an ultrasound imaging device 35 and a computing system 37 for performing processing steps. The ultrasound imaging device 35 is implemented as a hand-held unit to be positioned against the skin 4 of a subject's back. The hand-held unit can be moved along the skin 4 in a moving one-dimensional direction M substantially parallel to the lumbar spine 1. In the shown embodiment, the ultrasound imaging device 35 includes a marking unit 36 for marking an ultrasound imaging device location on the skin 4 on the lumbar spine 1 of the subject. The computing system 37 comprises a processor 38 for performing processing steps and a memory 39 for storing two-dimensional image data, e.g. high resolution image data such as a MRI, X-ray or CT based two-dimensional image data and/or ultrasound two-dimensional image data.

During use of the spinal navigation system 34, a MRI, X-ray or CT based two-dimensional image such as an X-ray image 20 of the lumbar spine 1 of the subject is provided as described above, preferably in DICOM format. In the present embodiment, an X-ray two-dimensional image 20 is generated in advance to the actual surgery process, e.g. a couple of weeks in advance providing a pre-operative image to facilitate a spinal navigation process. In order to generate the X-ray image 20, the subject is exposed to X-ray beams, usually in a separate X-ray image recording room using a dedicated apparatus, the room being provided with protecting means for protecting people from harmful X-ray beams.

Then, the hand-held ultrasound imaging device 35 is used to generate an ultrasound two-dimensional image 30 of the lumbar spine 1 of the subject. During ultrasound imaging, the subject can be present in the operating room, optionally even on the operating table, due to the hand-held feature of the ultrasound imaging device 35. The hand-held unit 35 includes a single or a multiple number of ultrasound transducers for emitting ultrasound waves and for receiving ultrasound waves that interacted with the lumbar spine 1 of the subject. In the shown embodiment, the ultrasound imaging device 35 is of the reflection type. In principle, however, also a transmission type ultrasound imaging device can be applied. The ultrasound two-dimensional image 30 is generated based on the emitted and received ultrasound waves. This process is either performed in the hand-held unit 35 or separately, e.g. in the computing system 37. Anyway, the ultrasound image 30 is made available to the computing system 37, e.g. via a transmission line 33, for further processing steps.

FIG. 4 shows an ultrasound image 30 of the subject's lumbar spine 1. Generally, the lumbar spine structures in the ultrasound image 30 are more fuzzy than in the corresponding X-ray image 20. In the shown embodiment, the marking unit 36 is visible in the ultrasound image 30.

As a next step, the processor 38 matches the ultrasound image 30 to the X-ray image 20 of the lumbar spine of the subject.

FIG. 5 shows a view wherein an X-ray image 20 and an ultrasound image 30 are matched. Here, the images 20 and 30 have been mirrored relative to the views shown in FIGS. 2 and 4. The left-hand side of FIG. 5 shows the X-ray image 20. According to an aspect of the invention, an X-ray spinal profile 41 following the exterior contour of a multiple number of spinal processus 3a-c on the X-ray image 20 is generated. Similarly, an ultrasound spinal profile 42 following the exterior contour of a multiple number of spinal processus on the ultrasound image 30 is generated. On the right-hand side of FIG. 5 both spinal profiles 41, 42 are shown. Then, the ultrasound spinal profile 42 is fitted to the X-ray spinal profile 41 by shifting the ultrasound spinal profile 42 in X-direction and/or Y-direction until the profiles have a maximum correlation, using some optimization scheme such as least squares.

It is noted that the X-ray spinal profile 41, or more generally, the MRI, X-ray or CT based spinal profile, as well as the corresponding ultrasound spinal profile 42 may include the complete spinal profile, or least parts corresponding to at least a part of the bony structures thereof, i.e. the processus or a number of consecutive processus. Preferably, the non-bony structure between the spinous processus are not used for correlating both spinal profiles 41, 42 since these spaces may vary because of a change in the patient pose. In case of multiple processus, the spinal profiles 41, 42 may be composed of a sequence of isolated consecutive processus contours. It is further noted that the spinal profiles 41, 42 can follow the exterior contour of a single spinal process or the exterior contour of a multiple number of spinal processes, such as specific spinal processes and/or subsequent spinal processes, thereby enhancing the reliability of the matching process.

The step of matching the MRI, X-ray or CT based two-dimensional image 20 to the ultrasound two-dimensional image 30 can be performed using numerical schemes, such as using Gaussian mixture models as described in the article “Robust Point Set Registration Using Gaussian Mixture Models” by Bing Jian et al. in IEEE Transactions on pattern analysis and machine intelligence, Vol. 33, No. 8, August 2011, pages 1633-1645.

Further, the step of generating a MRI, X-ray or CT spinal profile 41 and the step of generating an ultrasound spinal profile 42 can be performed using standard pattern recognition models, including segmentation, e.g. a so-called structure forest framework introduced for edge detection in natural images as described in the article “Structured Forest for Fast Edge Detection” by Piotr Dollar and C. Lawrence Zitnick in 2013 IEEE International conference on Computer Vision, pages 1841-1848, IEEE, December 2013. Alternatively, at least one of the profiles can be formed by the user, e.g. by using a user interface interacting with the respective image.

As a subsequent step, a pre-specified segment 21 of a spinal profile in the X-ray image 20 is related to a corresponding segment in the ultrasound two-dimensional image. Since, generally, the label or name of the respective processus is known, e.g. L3 or L4, it is then also known which processus in the ultrasound image are present, i.e. the same level, label or name can be assigned to the related processus in said ultrasound image. Then, it is known which vertebral body is being imaged by the ultrasound imaging device. The pre-specified segment 21 may include a particular spinal process, a number of particular spinal processus or a user-specified location as described below.

Preferably, the MRI, X-ray or CT based image includes a single or a multiple number of processus that are labeled such as L5 or S1. The processus included in the MRI, X-ray or CT based image can be labeled by identification, e.g. by a user of the system or automatically, e.g. based on pre-entered or library spinal data.

Preferably, the related segment in the ultrasound two-dimensional image may be visualized, e.g. by highlighting said segment, by displaying a pointer to said segment or by including label information to the ultrasound image. Additionally or alternatively, an audible, visible or tactile signal can be generated to inform the user that the pre-specified segment of a spinal profile in the MRI, X-ray or CT based image has been related to the corresponding segment in the ultrasound image.

Further, the method may comprises a step of relating the pre-specified segment of a spinal profile in the MRI, X-ray or CT based two-dimensional image to a position of the ultrasound imaging device 35 relative to the lumbar spine 1 of the subject. This can e.g. be performed by mapping the location of the marking unit 36 on the ultrasound imaging device 35, visible on the ultrasound image 30, see FIG. 4, or another reference point on the ultrasound image to a corresponding location 36′ on the X-ray image 20. Then, an offset D between said corresponding marking location 36′ and the pre-specified segment 21 can be determined, thereby determining a distance between the pre-specified segment 21 on the X-ray image 20 and the actual position of the ultrasound imaging device 35 relative to the spine 1 of the subject.

Alternatively, the surgeon may find other spinal levels by palpation and counting from a certain level that has been related to a known level in the MRI, X-ray or CT based image. The pre-specified segment 21 is preferably user-specified, e.g. by a surgeon specifying a desired entry location for surgery. In a preferred embodiment, the spinal navigation system 34 includes a user-interface for pre-specifying the segment 21 on the X-ray image 20, e.g. using a computer mouse. Further, the spinal navigation system 34 advantageously may include a display for displaying the X-ray image 20 and/or the ultrasound image 30. During the process, the X-ray image 20 can be depicted on the display and the surgeon may point out the place for entry, between two spinal processus, where it is desired to approach the lumbar spine.

In practice, the step of generating an ultrasound image, the step of matching the images and the step of relating the pre-specified segment 21 to the ultrasound imaging device location can repeatedly by performed, e.g. by moving the ultrasound imaging device 35 along the lumbar spine 1 in the moving direction M mainly parallel to the orientation of the lumbar spine 1. Then, the user of the spinal navigation system 34 may measure whether the imaging device 35 is moving to or away from the pre-specified or segment 21, in order to find said pre-specified segment 21.

In principle, the ultrasound image device can be moved or swiped along the one-dimensional direction M substantially parallel to the spine 1 until the distance D between the pre-specified segment 21 and the actual ultrasound imaging device position is smaller than a pre-defined offset value.

Since the ultrasound image is moved in a one-dimensional direction, the process of determining the offset D is a one-dimensional computational problem once the MRI, X-ray or CT based image an the ultrasound image have been matched. Then, the offset D can be computed relatively easily.

Further, an alerting signal can be generated when said distance is smaller than a pre-defined offset value, e.g. an audible, visible or tactile signal, in order to alert the user that the pre-specified segment 21 has been reached. Thus, an observation that the location of the ultrasound imaging device is close to or at the pre-specified spot can be performed by viewing the matched view on a display and/or automatically. By using the navigation system 34, the X-ray image 20 and the ultrasound image device 35 are used to project a pre-specified segment 21 on the skin 4 of the subject.

The pre-specified segment of a spinal profile in the MRI, X-ray or CT based two-dimensional image can be an exterior contour of a particular spinal process. Then, the user of the system may relate a specific spinous process, more specifically a level of said spinous process in the MRI, X-ray or CT based two-dimensional image to a corresponding segment in the ultrasound image, and, optionally, to a position of the ultrasound imaging device relative to the imaged spine of the subject. Also, a point of surgical entry between subsequent spinal processes may thus be determined.

Then, the user may use the marking unit 36 to mark the position on the skin 4 of the subject.

It is noted that the described method for spinal navigating can not only be used to indicate a point of surgical entry, but also to indicate a point of surgical passage or an identification point during surgery. As an example, a spinal navigating process can be applied when reaching the so-called fascie, i.e. a layer covering muscles and processus spinosus, in order to counteract that an incorrect direction deeper into the tissue is followed, especially with subjects having a relatively thick fat layer below the skin. In the latter case, palpation might not be possible. In the process of applying spinal navigation when approaching the fascia, the ultrasound imaging device will be draped in a sterile fashion. Then, the spinal navigating method can be used again, after an incision has been made in the patient's skin, e.g. to obtain better quality images and/or verify the position in case of obese patients, in order to counteract that a wrong correction is followed after opening the skin. The described spinal navigating method can be used as an alternative to presently known cumbersome methods including leaving a sterily draped C-arc in position and making further X-ray images when arrived at the layer of the fascia, injecting sterile ink and follow the ink path in further dissection to the fascia, or leaving a sterile lumbar needle in situ between the correct spinal processus

It is further noted that the matching step can be performed in various alternative manners. As an example, the two-dimensional X-ray and ultrasound image 20, 30 can be mapped to each other, partially or completely, in order to find an optimal correlation.

FIG. 6 shows a flow chart of an embodiment of a method according to the invention. The method is used for spinal navigation. The method comprises a step of providing 110 a MRI, X-ray or CT based two-dimensional image of the spine of a subject, a step of generating 120 an ultrasound two-dimensional image using an ultrasound imaging device on the spine of said subject, a step of matching 130 the ultrasound two-dimensional image to the MRI, X-ray or CT based two-dimensional image, and a step of relating 140 a pre-specified segment of a spinal profile in the MRI, X-ray or CT based two-dimensional image to a corresponding segment in the ultrasound two-dimensional image.

In practice, the method may be implemented by imaging the spinous processus both with X-ray and with ultrasound, retrieving the shape of the spinous process from the ultrasound image, e.g. by bone segmentation, and comparing the retrieved shape with the corresponding shapes of those structures in the X-ray image. In the X-ray image, the shape and the label, i.e. the name are known. Thus, by looking for the most similar shape in the X-ray, in a matching process, the level in the ultrasound image can be identified, based on the related corresponding level in the X-ray image so that it can be determined which level or label name for a vertebral body is currently imaged by the ultrasound device.

The method of performing spinal navigation can be performed using dedicated hardware structures, such as computer servers. Otherwise, the method can also at least partially be performed using a computer program product comprising instructions for causing a processor of a computer system or a control unit to perform a process including at least one of the method steps defined above. All (sub)steps can in principle be performed on a single processor. However, it is noted that at least one step can be performed on a separate processor, e.g. the step of matching the ultrasound image to the MRI, X-ray or CT based image. A processor can be loaded with a specific software module. Dedicated software modules can be provided, e.g. from the Internet.

The invention is not restricted to the embodiments described herein. It will be understood that many variants are possible.

In this context it is noted that the invention can not only be applied in case of a slipped, herniated intervertebral disc or HNP, but also for other lumbar spine surgery such as lumbar spinal stenosis and intra-spinal tumors, or more general to spine surgery such as thoracic or cervical spine surgery.

It is further noted that, within the context of the application, an MRI, X-ray or CT based two-dimensional image or an ultrasound two-dimensional image can be a image of the entire spine or a portion thereof, such as a two-dimensional image of the lumbar part of the spine.

These and other embodiments will be apparent for the person skilled in the art and are considered to fall within the scope of the invention as defined in the following claims For the purpose of clarity and a concise description features are described herein as part of the same or separate embodiments. However, it will be appreciated that the scope of the invention may include embodiments having combinations of all or some of the features described.

1. A spinal navigation method, comprising the steps of:
providing a MRI, X-ray or CT based two-dimensional image of the spine of a subject;
generating an ultrasound two-dimensional image using an ultrasound imaging device on the spine of said subject;
matching the ultrasound two-dimensional image to the MRI, X-ray or CT based two-dimensional image, and
relating a pre-specified segment of a spinal profile in the MRI, X-ray or CT based two-dimensional image to a corresponding segment in the ultrasound two-dimensional image.

2. The spinal navigation method according to claim 1, wherein the step of matching the ultrasound image to the MRI, X-ray or CT based two-dimensional image includes the sub-steps of:
generating a MRI, X-ray or CT spinal profile following the exterior contour of a multiple number of spinal processus on the MRI, X-ray or CT based two-dimensional image;
generating an ultrasound spinal profile following the exterior contour of a multiple number of spinal processus on the ultrasound image, and
fitting the ultrasound spinal profile to the MRI, X-ray or CT spinal profile.

3. The spinal navigation method according to claim 1, wherein the MRI, X-ray or CT based image includes a single or a multiple number of processes that are labelled.

4. The spinal navigation method according to claim 1, further comprising visualizing the segment in the ultrasound image that relates to the corresponding pre-specified segment in the MRI, X-ray or CT based image.

5. The spinal navigation method according to claim 1, wherein the generating step, the matching step and the relating step are repeatedly performed.

6. The spinal navigation method according to claim 1, further comprising a step of relating the pre-specified segment of a spinal profile in the MRI, X-ray or CT based two-dimensional image to a position of the ultrasound imaging device relative to the spine of the subject.

7. The spinal navigation method according to claim 6, wherein the relating step includes determining a distance between the pre-specified segment and an actual ultrasound imaging device position relative to the spinal spine of the subject.

8. The spinal navigation method according to claim 6, including the step of moving the ultrasound imaging device on the spinal spine and repeatedly relating the ultrasound imaging device position to the pre-specified segment.

9. The spinal navigation method according to claim 8, wherein the moving step is performed until the distance between the pre-specified segment and the actual ultrasound imaging device position is smaller than a pre-defined offset value.

10. The spinal navigation method according to claim 8, including the step of generating an alerting signal when the distance between the pre-specified segment and the actual ultrasound imaging device position is smaller than a pre-defined offset value.

11. The spinal navigation method according to claim 1, wherein the pre-specified segment of a spinal profile in the MRI, X-ray or CT based two-dimensional image is an exterior contour of a spinal process or a spot between subsequent spinal processes.

12. The spinal navigation method according to claim 1, further comprising the step of marking the position of the ultrasound imaging device on the skin on the spinal spine of the subject.

13. A spinal navigation system, comprising an ultrasound imaging device for generating an ultrasound two-dimensional image of the spine of a subject, and a computing system for performing the steps of:
providing a MRI, X-ray or CT based two-dimensional image of the spine of the subject;
matching the ultrasound two-dimensional image to the MRI, X-ray or CT based two-dimensional image of the spine of the subject, and
relating a pre-specified segment of a spinal profile in the MRI, X-ray or CT image to a corresponding segment in the ultrasound two-dimensional image.

14. The spinal navigation system according to claim 13, wherein the ultrasound imaging device is a hand-held unit.

15. The spinal navigation system according to claim 13, wherein the ultrasound imaging device includes a marking unit for marking an ultrasound imaging device location on the skin on the lumbar spine of the subject.

16. The spinal navigation system according to claim 13, further including a user-interface for pre-specifying the segment on the MRI, X-ray or CT image.

17. The spinal navigation system according to claim 13, further including a display for displaying the MRI, X-ray or CT two-dimensional image and/or the ultrasound image.

18. A computer program product for spinal navigation, the computer program product comprising computer readable code for causing a processor to perform a process including the steps of:
providing a MRI, X-ray or CT based two-dimensional image of the spine of the subject;
generating an ultrasound two-dimensional image using an ultrasound imaging device on the spine of said subject;
matching the ultrasound two-dimensional image to the MRI, X-ray or CT based two-dimensional image, and
relating a pre-specified segment of a spinal profile in the MRI, X-ray or CT image to a corresponding segment in the ultrasound two-dimensional image.
